(19) United States
(12) Patent Application Publication
(10) Pub. No.: US 2010/0119612 A1
(43) Pub. Date: May 13, 2010

(54) NANOPARTICLES COMPRISING NON-CRYSTALLINE DRUG

(75) Inventors: Dwayne Thomas Friesen, Bend, OR (US); Daniel Tod Smithey, bend, OR (US); Michael Mark Morgen, Bend, OR (US); Ralph Tadday, Bend, OR (US)

Correspondence Address: CHERNOFF, VILHAUER, MCCLUNG & STENZEL, LLP, 601 SW Second Avenue, Suite 1600, PORTLAND, OR, 97204-3157, US

(73) Assignee: Bend Research, Inc

(21) Appl. No.: 12/450,563

(22) PCT Filed: Apr. 7, 2008
(86) PCT No.: PCT/IB2008/000844

§ 371 Date: Sep. 30, 2009

Related U.S. Application Data
(60) Provisional application No. 60/912,228, filed on Apr. 17, 2007.

(51) Int. Cl. A61K 9/51; A61K 31/47

(52) U.S. Cl. 424/498; 514/313

(57) ABSTRACT

A pharmaceutical composition comprises nanoparticles comprising a core of non-crystalline drug and surface stabilizers consisting of a phospholipid and a bile salt.

### BACKGROUND OF THE INVENTION

The present invention relates to nanoparticles comprising non-crystalline drug, a phospholipid and a bile salt.

A variety of approaches to solubilize poorly water soluble drugs have been developed, including liposomes and nanoparticles. Liposomes are formed when phospholipids are dispersed in an aqueous medium. When dispersed gently, they swell, hydrate, and spontaneously form multilamellar, concentric, bilayer vesicles with layers of aqueous media separating the lipid bilayers. These systems commonly are referred to as multilamellar liposomes, or multilamellar vesicles, and have diameters from 25 nm to 4 microns. Sonication or solvent dilution of multilamellar vesicles results in the formation of small unilamellar vesicles with diameters in the range of from 30 to 50 nm, containing an aqueous solution in the core. Liposomes have been used as carriers for drugs, since water- or lipid-soluble substances can be entrapped in the aqueous spaces or within the bilayer itself, respectively. However, liposomes have the disadvantage that maximum drug loading is limited for most drugs, they are often not physically stable in the hydrated form, and are difficult to resuspend if dried.

It is known that poorly water soluble drugs may be formulated as nanoparticles. Nanoparticles are of interest for a variety of reasons, such as to improve the bioavailability of poorly soluble drugs, to provide targeted drug delivery to specific areas of the body, to reduce side effects, or to reduce variability in vivo.

Several approaches have been taken to formulate drugs as nanoparticles. One approach is to decrease the size of crystalline drug by grinding or milling the drug in the presence of a surface modifier. See, e.g., Liversidge, et al., U.S. Pat. No. 5,145,684. Another approach to forming nanoparticles is to precipitate the drug in the presence of a film forming material such as a polymer. See, e.g., Fessi et al., U.S. Pat. No. 5,118,528. Violante at al., U.S. Pat. No. 4,826,689 disclose a method for making nanoparticles of amorphous drug by infusing an aqueous solution into an organic solution in which is dissolved a water-insoluble drug.

Lipids, including phospholipids, have been used as excipients to make small drug particles. For example, Speiser, U.S. Pat. No. 4,880,634 discloses an aqueous suspension excipient system comprising nano-pellets comprising a lipid and a surfactant. The nano-pellets are formed by melting the lipid or lipid mixture, heating water to the same temperature as the melting point of the lipid, adding the drug to the lipid, and then adding the warm aqueous phase and thoroughly mixing.

Haynes, U.S. Pat. No. 5,091,187 discloses an injectable composition for water insoluble drugs as an aqueous suspension of phospholipid coated microcrystals. The microcrystals are formed by sonication or other treatment involving high shear in the presence of lecithin or other membrane forming lipid. A secondary coating of phospholipids may be added after the initial sonication step.

Domb, U.S. Pat. No. 5,188,837 discloses lipospheres for controlled delivery. The lipospheres are solid water insoluble microparticles that have a layer of phospholipid embedded on their surface. The core of the liposphere is a solid substance to be delivered. The particles range in size from about 0.3 to 250 microns.

Sjostrom, et al., Journal of Pharmaceutical Sciences, Vol. 82, No. 6 June 1993, pages 584-589, discloses nanoparticles formed from a variety of materials, including phospholipids and bile salts. The model compound cholesterol acetate used in these nanoparticles crystallized during formation of the nanoparticles.

There remain a number of problems associated with the use of nanoparticles to deliver pharmaceutical compounds to the body. First, it is difficult to form very small particles. Second, once particles of the target size are formed, they must remain stable over time in a variety of different environments. The nanopartides must be stabilized so that they do not aggregate into larger particles. Often, the nanoparticles are formed in a liquid environment. The nanoparticles should be capable of being dried to a solid form which may be stored. The nanoparticles must also be capable of being reconstituted for administration to the body, and remain stable in vivo.

Third, the nanoparticles must be well tolerated in the body. Often surface modifiers such as surfactants are used to stabilize the nanoparticles, but such materials can have adverse physiological effects when administered in vivo.

Finally, the nanoparticles must be formulated to provide optimum delivery. The nanoparticles should provide good bioavailability. In some applications, it is desired that the nanopartides provide rapid onset, or reduce fed/fasted effects if administered orally. Finally, it may be desired to administer the nanoparticles through non-oral routes, such as for parenteral, topical, or ocular delivery.

Accordingly, there is still a continuing need for nanoparticles that are stable, in the sense of not aggregating into larger particles, and that provide for high concentrations of dissolved drug for sustained periods of time so as to improve bioavailability.

### BRIEF SUMMARY OF THE INVENTION

In a first aspect, a pharmaceutical composition comprises nanoparticles comprising a poorly water soluble drug, in which at least 90 wt % of the drug is non-crystalline. The nanoparticles comprise one or more phospholipids and one or more bile salts present in a weight ratio of from 1:0.05 to 1:4 (wt phospholipid:wt bile salt). The nanoparticles have an average size of less than 500 nm. The drug, the phospholipid(s), and the bile salt(s) collectively constitute at least 80 wt % of the nanoparticles. The nanoparticles comprise a core comprising the drug surrounded by a layer of phospholipid and bile salt. The drug has a LogP of greater than 4, and at least one of the following: (1) a melting temperature of less than 110° C. and (2) a glass transition temperature (T_{g}) of greater than 40° C.

In another aspect, a process is provided for forming nanoparticles. A poorly water soluble drug is dissolved in an organic solvent to form an organic solution. The drug has a LogP of greater than 4, and at least one of the following: (1) a melting temperature of less than 110° C.; and (2) a glass transition temperature (T_{g}) of greater than 40° C. An emulsion is formed comprising the organic solution, a non-solvent and a phospholipid and a bile salt present in a weight ratio of from 1:0.05 to 1:4 (wt phospholipid:wt bile salt). The drug is poorly soluble in the non-solvent and the organic solvent is immiscible in the non-solvent. The organic solvent is removed to form a suspension of nanoparticles having an average size of less than 500 nm, wherein at least 90 wt % of the drug in the nanoparticles is non-crystalline, and the drug, the phospholipid, and the bile salt constitute at least 80 wt % of the nanoparticles.

In another aspect, a pharmaceutical composition comprises nanoparticles comprising a non-crystalline cholesterol ester transfer protein (CETP) inhibitor and one or more surface stabilizers, the nanoparticles having a diameter of less than 500 nm.

The nanoparticles of the present invention provide a number of advantages, First, nanoparticles comprising non-crystalline drug, phospholipid, and bile salt are capable of providing high levels of free drug (described below) and hence greater bioavailability. This is believed to be due to the non-crystalline nature of the drug and the small size of the particles.

Second, the nanoparticles also provide good physical stability of the non-crystalline drug due to the use of drugs which are hydrophobic (characterized by a LogP greater than 4) and which have either a low melting temperature (less than 110° C.) or a high glass transition temperature (greater than 40° C.). The inventors have found that such drugs with such properties are capable of being formulated into stable nanoparticles of non-crystalline drug. Without wishing to be bound by any particular theory, it is believed that the tendency of a drug to change from the non-crystalline (or amorphous) form to crystalline form is related to its melting temperature, its glass transition temperature and its hydrophobicity (characterized by its LogP). The physical stability of the non-crystalline form of the drug in aqueous environments tends to increase as the melt temperature decreases, the glass transition temperature increases and the hydrophobicity increases.

Another advantage of the nanoparticles is the use of phospholipids and bile salts as surface stabilizers. The nanoparticles consist of a core of non-crystalline drug surrounded by the phospholipid and bile salt, which act as surface stabilizers. These materials are well tolerated in vivo, and provide reduced toleration issues relative to other surface stabilizers. In addition, the combination of the phospholipids and bile salts has the advantage that very small nanoparticles can be formed, often less than 100 nm. The bile salt provides ionizable groups. Such groups are capable of being charged in a use environment, which helps to reduce aggregation of the particles when suspended in solution. The phospholipid in turn provides a “template” for the bile salt to intercalate. This reduces the amount of bile salt required to form a stable nanoparticle in suspension.

Yet another advantage of the nanoparticles is that they consist primarily of the drug, phospholipid(s) and bile salt(s). The nanoparticles do not require the use of an additional solubilizing oil, fat or wax in which to incorporate the drug, and thus can obtain higher drug loadings. In addition, the nanoparticles can achieve faster release of drug without the presence of a fat or wax.

The foregoing and other objectives, features, and advantages of the invention will be more readily understood upon consideration of the following detailed description of the invention.

### BRIEF DESCRIPTION OF THE SEVERAL VIEWS OF THE DRAWINGS

FIG. 1 is a differential scanning calorimetry trace of the nanoparticles of Example 2.

FIG. 2 is a powder x-ray diffraction pattern of the nanoparticles of Example 2.

### DETAILED DESCRIPTION OF THE INVENTION

The nanoparticles of the present invention comprise a poorly water soluble drug, one or more phospholipids, and one or more bile salts. By “nanoparticles” is meant a plurality of small particles having an average size of less than 500 nm. When measured in a suspension, “average size” means the effective cumulant diameter as measured by dynamic light scattering (DLS), using for example, Brookhaven Instruments' 90Plus particle sizing instrument. Preferably, the average size of the nanoparticles is less than 400 nm, more preferably less 300 nm, even more preferably less than 200 nm, and most preferably less than 100 nm. The nanoparticles do not include a phospholipid bilayer, and thus are not liposomes, micelles, or vesicles, and do not include a solubilizing oil and thus are not microemulsions or emulsion droplets.

The width of the particle size distribution in a suspension is given by the “polydispersity” of the particles, which is defined as the relative variance in the correlation decay rate distribution, as is known by one skilled in the art. See B. J. Fisken, “Revisiting the method of cumulants for the analysis of dynamic light-scattering data,” Applied Optic. Preferably, the polydispersity of the nanoparticles is less than 0.7. More preferably, the polydispersity of the nanoparticles is less than about 0.5, and more preferably less than 0.3. In one embodiment, the average size of the nanoparticles is less than 500 nm with a polydispersity of 0.5 or less. In another embodiment, the average size of the nanoparticles is less than 300 nm with a polydispersity of 0.5 or less.

The presence of nanoparticles in a solid composition of the present invention can be determined using the following procedure. A sample of the solid composition is embedded in a suitable material, such as an epoxy or polyacrylic acid (e.g., LR White from London Resin Co., London, England). The sample is then microtomed to obtain a cross-section of the solid composition that is about 100 to 200 nm thick. This sample is then analyzed using transmission electron microscopy (TEM) with energy dispersive X-ray (EDX) analysis. TEM-EDX analysis quantitatively measures the concentration and type of atoms larger than boron over the surface of the sample. From this analysis, regions that are rich in drug can be distinguished from regions that are rich in other materials. The size of the regions that are rich in drug will have an average diameter of less than 500 nm in this analysis, demonstrating that the solid composition comprises nanoparticles of drug. See, for example, Transmission Electron Microscopy and Diffractometry of Materials (2001) for further details of the TEM-EDX method.

Another procedure that demonstrates the solid composition contains nanoparticles is to administer a sample of the solid composition to water to form a suspension of the nanopartides. The suspension is then analyzed by DLS as described above. A solid composition of the invention will form nanoparticles having an average cumulant diameter of less than 500 nm.

A specific procedure for demonstrating the solid composition contains nanoparticles is as follows. A sample of the solid composition is added to water at ambient temperature at a concentration of up to 1 mg/mL. The so-formed suspension is then analyzed by DLS. The solid composition contains nanoparticles if the DLS analysis results in particles having an average cumulant diameter of less than 500 nm.

A solid composition of the invention will show the presence of nanoparticles in at least one, and preferably both of the above tests.

At least 90 wt % of the poorly water soluble drug in the nanopartides is non-crystalline. Preferably at least about 95 wt % of the drug in the nanoparticle is non-crystalline; in other words, the amount of drug in crystalline form does not exceed about 5 wt %. Amounts of crystalline drug may be measured by Powder X-Ray Diffraction (PXRD), by Scanning Electron Microscope (SEM) analysis, by differential scanning calorimetry (DSC), or by any other known quantitative measurement.

The drug is a poorly water soluble drug. By “poorly water soluble” is meant that the drug has a minimum aqueous solubility over the pH range of 6.5 to 7.5 of about 1 mg/mL or less. The drug may have an even lower aqueous solubility, such as less than about 0.5 mg/mL, less than about 0.1 mg/mL, and even less than about 0.01 mg/mL over the pH range of 6.5 to 7.5.

Preferred classes of drugs include, but are not limited to, antihypertensives, antianxiety agents, anticlotting agents, anticonvulsants, blood glucose-lowering agents, decongestants, antihistamines, antitussives, antineoplastics, antiarrythmics, beta blockers, anti-inflammatories, antipsychotic agents, cognitive enhancers, anti-atherosclerotic agents, cholesterol-reducing agents, triglyceride-reducing agents, antiobesity agents, autoimmune disorder agents, anti-impotence agents, antibacterial and antifungal agents, hypnotic agents, anti-Parkinsonism agents, anti-Alzheimer's disease agents, antibiotics, anti-depressants, antiviral agents, glycogen phosphorylase inhibitors, cholesteryl ester transfer protein (CETP) inhibitors, and microsomal Triglyceride Transfer Protein inhibitor (MTP inhibitor), microsomal triglyceride transfer protein (MTP) inhibitors, anti-angiogenesis agents, vascular endothelial growth factor (VEGF) receptor inhibitors, and carbonic anhydrase inhibitors.

The drug is physically stable in the non-crystalline state, meaning that the drug does not readily crystallize from the non-crystalline state. The inventors have found that one important physical property of drugs which are stable in the non-crystalline state is that the drugs are hydrophobic. By hydrophobic is meant that the Log P of the drug is at least 4. The LogP of the drug may be at least 5, at least 6, or even at least 7. Log P, defined as the base 10 logarithm of the ratio of (1) the drug concentration in an octanol phase to (2) the drug concentration (in its unionized form if the drug may be ionized) in a water phase when the two phases are in equilibrium with each other, is a widely accepted measure of hydrophobicity. Log P may be measured experimentally or calculated using methods known in the art. When using a calculated value for Log P, the highest value calculated using any generally accepted method for calculating Log P is used. Calculated Log P values are often referred to by the calculation method, such as Clog P, Alog P, and Mlog P. The Log P may also be estimated using fragmentation methods, such as Crippen's fragmentation method (27 J. Chem. Inf. Comput. Sci. 21 (1987)); Viswanadhan's fragmentation method (29 J. Chem. Inf. Comput. Sci. 163 (1989)); or Broto's fragmentation method (19 Eur. J. Med. Chem.-Chim. Theor. 71 (1984)). Preferably the Log P value is calculated by using the average value estimated using Crippen's, Viswanadhan's, and Broto's fragmentation methods.

To form nanoparticles comprising non-crystalline drug, the drug should also have either a Tₘ of less than 110° C., or a T_{g} of greater than 40° C. Drugs that meet at least one of these two properties tend to be stable in the non-crystalline state.

In one embodiment, the Tₘ of the drug is less than 110° C. Without wishing to be bound by theory, it is believed that the driving force for crystallization tends to increase as the melting point, Tₘ, increases. Accordingly, the invention has increased utility as the Tₘ of the drug decreases. The Tₘ of the drug may be less than 105° C., less than 100° C., less than 95° C., or even less than 90° C.

In another embodiment, the T_{g} of the drug is greater than 40° C. Without wishing to be bound by theory, it is believed that the kinetic barrier to crystallization tends to increase as the glass transition temperature, T_{g}, increases. Accordingly, the invention has increased utility as the T_{g} of the drug increases. The T_{g} of the drug may be greater than 45° C., greater than 50° C., greater than 55° C., or even greater than 60° C. As used herein, T_{g} refers to the T_{g} of the drug alone measured in the solid state at less than 5% relative humidity.

Another important property of drugs which tend to be physically stable in the non-crystalline state is the relative values of the melting temperature Tₘ and glass transition temperature T_{g} of the drug. Without wishing to be bound by any particular theory, it is believed that the tendency for a drug to crystallize tends to increase as the ratio of the Tₘ of the drug (in K) to the glass-transition temperature for the drug, T_{g} (in K), increases. The driving force for crystallization is dominated by the melting point Tₘ, while the kinetic barrier to crystallization is controlled primarily by the T_{g}. The ratio, Tₘ/T_{g} (in K/K), indicates the relative propensity for a drug to crystallize. Since the stability of the drug in the non-crystalline state (that is, its tendency to crystallize) increases as the ratio Tₘ/T_{g} decreases, the invention finds increasing utility with decreasing Tₘ/T_{g}. In general, the Tₘ/T_{g} value should be less than 1.35. Preferably, the Tₘ/T_{g} value is less than 1.3, more preferably less than 1.25, and most preferably less than 1.2.

The nanoparticles comprise a core comprising non-crystalline drug (which may be liquid or solid) surrounded by a layer comprising the phospholipid and bile salt. The core is primarily drug, meaning that at least 50 wt % of the core is drug. More preferably, at least 75 wt % of the core is drug, and even more preferably at least 90 wt % of the core is drug. In one embodiment, the core consists essentially of the non-crystalline drug. In one embodiment, the cores are substantially free from an aqueous phase.

Nanoparticles having a single drug glass transition temperature are considered to comprise such cores consisting of non-crystalline drug. Nanoparticles having a solid core of essentially non-crystalline drug are preferred as the reduced mobility of the molecular drug species within the solid core is believed to lead to a more physically stable particle, especially with regard to coalescence. By solid core is meant that the T_{g} of the drug in the core is greater than 20° C.

CETP inhibitors are a preferred class of drugs since these drugs are generally very poorly water soluble, very hydrophobic (Log P>4), and have low ratios of Tₘ/T_{g} (of less than 1.35). CETP inhibitors are drugs that inhibit CETP activity. The effect of a drug on the activity of CETP can be determined by measuring the relative transfer ratio of radiolabeled lipids between lipoprotein fractions, essentially as previously described by Morton in J. Biol. Chem. 256, 11992, 1981 and by Dias in Clin. Chem. 34, 2322, 1988, and as presented in U.S. Pat. No. 6,197,786, the disclosures of which are herein incorporated by reference. The potency of CETP inhibitors may be determined by performing the above-described assay in the presence of varying concentrations of the test compounds and determining the concentration required for 50% inhibition of transfer of radiolabeled lipids between lipoprotein fractions. This value is defined as the “IC₅₀ value.” Preferably, the CETP inhibitor has an IC₅₀ value of less than about 2000 nM, more preferably less than about 1500 nM, even more preferably less than about 1000 nM, and most preferably less than about 500 nM.

Specific examples of CETP inhibitors include [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester (torcetrapib), [2R,4S] 4-[acetyl-(3,5-bis-trifluoromethyl-benzyl)-amino]-2-ethyl-6-trifluoromethyl-3,4-dihydro-2H-quinoline-1-carboxylic acid isopropyl ester, [2R, 4S] 4-[(3,5-bis-trifluoromethyl-benzyl)-methoxycarbonyl-amino]-2-ethyl-6-trifluoromethyl-3,4-dihydro-2H-quinoline-1-carboxylic acid isopropyl ester, (2R)-3-[[3-(4-chloro-3-ethylphenoxy)phenyl][[3-(1,1,2,2-tetrafluoroethoxy)phenyl]methyl]amino]-1,1,1-trifluoro-2-propanol, (2R, 4R, 4aS)-4-[amino-(3,5-bis-(trifluoromethyl-phenyl)-methyl]-2-ethyl-6-(trifluoromethyl)-3,4-dihydroquinoline-1-carboxylic acid isopropyl ester, S-[2-([[1-(2-ethylbutyl)cyclohexyl]carbonyl]amino)phenyl]2-methylpropanethioate, trans-4-[[[2-[[[[3,5-bis(trifluoromethyl)phenyl]methyl](2-methyl-2H-tetrazol-5-yl)amino]methyl]-4-(trifluoromethyl)phenyl]ethylamino]methyl]-cyclohexaneacetic acid, trans-(4-{[N-(2-{[N′-[3,5-bis(trifluoromethyl)benzyl]-N′-(2-methyl-2H-tetrazol-5-yl)amino]methyl}-5-methyl-4-trifluoromethylphenyl)-N-ethylamino]methyl}cyclohexyl)acetic acid methanesulfonate, trans-(2R,4S)-2-(4-{4-[(3 15-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetamide, methyl N-(3-cyano-5-trifluoromethylbenzyl)-[6-(N-cyclopentylmethyl-N-ethylamino)indan-5-ylmethyl]-carbamate, methyl (3-cyano-5-trifluoromethylbenzyl)-[6-(N′-cyclopentylmethyl-N′-ethylamino)indan-5-ylmethyl]-carbamate, ethyl 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carboxylate, tert-butyl 5-(N-(3,5-bis(trifluoromethyl)benzyl)acetamido)-7-methyl-8-(trifluoromethyl)-2,3,4,5-tetrahydrobenzo[b]azepine-1-carboxylate, the drugs disclosed in commonly owned U.S. patent application Ser. Nos. 09/918,127 and 10/066,091, the disclosures of both of which are incorporated herein by reference, and the drugs disclosed in the following patents and published applications, the disclosures of all of which are incorporated herein by reference: DE 19741400 A1; DE 19741399 A1; WO 9914215 A1; WO 9914174; DE 19709125 A1; DE 19704244 A1; DE 19704243 A1; EP 818448 A1; WO 9804528 A2; DE 19627431 A1; DE 19627430 A1; DE 19627419 A1; EP 796846 A1; DE 19832159; DE 818197; DE 19741051; WO 9941237 A1; WO 9914204 A1; JP 11049743; WO 0018721; WO 0018723; 9WO 0018724; WO 0017164; WO 0017165; WO 0017166; EP 992496; EP 987251; WO 9835937; JP 03221376; WO 04020393; WO 05095395; WO 05095409; WO 05100298; WO 05037796; WO 0509805; WO 03028727; WO 04039364; WO 04039453; and WO 0633002.

Thus, in one embodiment, the CETP inhibitor is selected from the group consisting of torcetrapib, [2R,4S] 4-[acetyl-(3,5-bis-trifluoromethyl-benzyl)-amino]-2-ethyl-6-trifluoromethyl-3,4-dihydro-2H-quinoline-1-carboxylic acid isopropyl ester, [2R, 4S] 4-[(3,5-bis-trifluoromethyl-benzyl)-methoxycarbonyl-amino]-2-ethyl-6-trifluoromethyl-3,4-dihydro-2H-quinoline-1-carboxylic acid isopropyl ester, (2R)-3-[[3-(4-chloro-3-ethylphenoxy)phenyl][[3-(1,1,2,2-tetrafluoroethoxy)phenyl]methyl]amino]-1,1,1-trifluoro-2-propanol, (2R, 4R, 4aS)-4-[amino-(3,5-bis-(trifluoromethyl-phenyl)-methyl]-2-ethyl-6-(trifluoromethyl)-3,4-dihydroquinoline-1-carboxylic acid isopropyl ester, S-[2-([[1-(2-ethylbutyl)cyclohexyl]carbonyl]amino)phenyl]2-methylpropanethioate, trans-4-[[[2-[[[[3,5-bis(trifluoromethyl)phenyl]methyl](2-methyl-2H-tetrazol-5-yl)amino]methyl]-4-(trifluoromethyl)phenyl]ethylamino]methyl]-cyclohexaneacetic acid, Trans-(4-{[N-(2-{[N′-[3,5-bis(trifluoromethyl)benzyl]-N′-(2-methyl-2H-tetrazol-5-yl)amino]methyl}-5-methyl-4-trifluoromethylphenyl)-N-ethylamino]methyl}cyclohexyl)acetic acid methanesulfonate, trans-(2R,4S)-2-(4-(4-{4-[(3 15-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetamide, methyl N-(3-cyano-5-trifluoromethylbenzyl)-[6-(N-cyclopentylmethyl-N-ethylamino)indan-5-ylmethyl]-carbamate, methyl (3-cyano-5-trifluoromethylbenzyl)-[6-(N′-cyclopentylmethyl-N′-ethylamino)indan-5-ylmethyl]carbamate, ethyl 4-(3,5-bis(trifluoromethyl)phenyl)(2-methyl-2H-tetrazol-5-yl)methyl)-2-ethyl-6-(trifluoromethyl)-3,4-dihydroquinoxaline-1(2H)-carboxylate, and tert-butyl 5-(N-(3,5-bis(trifluoromethyl)benzyl)acetamido)-7-methyl-8-(trifluoromethyl)-2,3,4,5-tetrahydrobenzo[b]azepine-1-carboxylate.

In another embodiment, the CETP inhibitor is torcetrapib.

In still another embodiment, the CETP inhibitor is (2R)-3-[[3-(4-chloro-3-ethylphenoxy)phenyl][[3-(1,1,2,2-tetrafluoroethoxy)phenyl]methyl]amino]-1,1,1-trifluoro-2-propanol.

In still another embodiment, the CETP inhibitor is trans-(2R,4S)-2-(4-{4-[(3 15-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexylyacetamide.

The nanoparticles comprise surface stabilizers consisting of a mixture of one or more phospholipids and one or more bile salts. These surface stabilizers are chosen to reduce aggregation or flocculation of the particles in an aqueous solution.

The term “phospholipid” includes both naturally occurring and synthetic phospholipids, as well as mixtures of phospholipids. Phospholipids that may be used include phosphatidic acids, phosphatidyl cholines, phosphatidyl ethanolamines, phosphatidylglycerols, phosphatidylserines, phosphatidylinositols, lysophosphatidyl derivatives, and cardiolipin.

The term phospholipid includes “lecithin”, which refers to mixtures of phospholipids obtained from plant and animal sources. Commercially available lecithin is obtained from egg yolk, soybeans, and other plant and animal products. The composition of lecithin varies depending upon the source. Egg lecithin may contain about 69% phosphatidylcholine and 24% phosphatidylethanolamine, as well as other components. Soybean lecithin may contain about 21% phosphatidylcholine, 22% phosphatidylethanolamine, and 19% phosphatidyllinositol, as well as other components. A preferred lecithin is egg yolk lecithin.

A preferred phospholipid is 1-2-diacylphosphotidylcholine, which refers generally to phosphatidylcholine having two fatty acids linked to the glycerol. The fatty acids may be the same or different, and may be saturated or unsaturated. Exemplary saturated fatty acids include lauric, myristic, palmitic and stearic acid. Exemplary unsaturated fatty acids include oleic, linoleic and linolenic acid.

Examples of specific phosphatidylcholines include dioleoylphosphatidylcholine, dimyristoylphosphatidylcholine (DMPC), dipentadecanoylphosphatidylcholine, dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), dilignoceroylphosphatidylcholine (DLPC), 1-myristoyl-2-oleoyl-sn-glycero-3-phosphocholine, 1-O-palmityl-2-acetyl-rac-glycero-3-phosphocholine, 1-O-palmityl-2-arachidonoyl-sn-glycero-3-phosphocholine, 1-O-palmityl-2-(cis-8,11,14-eicosatrienoyl)-sn-glycero-3-phosphocholine, 1-O-palmityl-2-O-methyl-rac-glycero-3-phosphocholine, 1-O-palmityl-2-palmitoyl-rac-glycero-3-phosphocholine, 1-oleoyl-2-palmitoyl-sn-glycero-3-phosphocholine, 1-palmitoyl-2-(cis-4,7,10,13,16,19-docosahexaenoyl)-sn-glycero-3-phosphocholine, 1-palmitoyl-2-(pyrene-1-yl)decanoyl-sn-glycero-3-phosphocholine, 1-stearoyl-2-linoleoyl-sn-glycero-3-phosphocholine, 2-arachidonoyl-1-palmitoyl-sn-glycero-3-phosphocholine, 2-arachidonoyl-1-stearoyl-sn-glycero-3-phosphocholine, and 2-oleoyl-1-stearoyl-sn-glycero-3-phosphocholine. A preferred phosphatidylcholine is 1-oleoyl-2-palmitoyl-sn-glycero-3-phosphocholine.

Examples of phosphatidylethanolamines include dicaprylphosphatidylethanolamine, dioctanoylphosphatidylethanolamine, dilauroylphosphatidylethanolamine, dlmyristoylphosphatidylethanolamine (DMPE), dipalmitoylphosphatidylethanolamine (DPPE), dipalmitoleoylphosphatidylethanolamine, distearoylphosphatidylethanolamine (DSPE), dioleoylphosphatidylethanolamine, and dilineoylphosphatidylethanolamine.

Examples of phosphatidylglycerols include dicaprylphosphatidylglycerol, dioctanoylphosphatidylglycerol, dilauroylphosphatidylglycerol, dimyristoylphosphatidylglycerol (DMPG), dipalmitoylphosphatidylglycerol (DPPG), dipalmitoleoylphosphatidylglycerol, distearoylphosphatidylglycerol (DSPG), dioleoylphosphatidylglycerol, and dilineoylphosphatidylglycerol.

Additional examples of phospholipids include modified phospholipids, for example phospholipids having their head group modified, e.g., alkylated or polyethylene glycol (PEG)-modified, hydrogenated phospholipids, phospholipids with a variety of head groups (phosphatidylmethanol, phosphatidylethanol, phosphatidylpropanol, phosphatidylbutanol, etc.), dibromo phosphatidylcholines, mono and diphytanoly phosphatides, and mono and diacetylenic phosphatides. Synthetic phospholipids with asymmetric acyl chains (e.g., with one acyl chain of 6 carbons and another acyl chain of 12 carbons) may also be used.

Bile salts are the acid addition salts of bile acids. The bile acids are divided into two groups: primary (derived from cholesterol) and secondary (derived from primary bile acids). The bile salts are conjugated through peptide linkages to glycine or taurine. The primary bile salts are taurine or glycine conjugates of cholic acid or chenic acid; the secondary bile salts are taurine and glycine conjugates of deoxycholic and lithocholic acids. See Remington the Science and Practice of Pharmacy (20^{th} edition, 2000, at page 1228). The term “bile salt” includes mixtures of bile salts.

Exemplary bile salts include the salts of dihydroxy cholic acids, such as deoxycholic acid, glycodeoxycholic acid, taurodeoxycholic acid, chenodeoxycholic acid, glycochenodeoxycholic acid, and taurochenodeoxycholic acid, and trihydroxy cholic acids, such as cholic acid, glycocholic acid, and taurocholic acid. The acid addition salts include sodium, and potassium. Preferred bile salts include sodium glycocholate and sodium taurocholate.

Exemplary mixtures of phospholipid and bile salt include 1,2-diacylphosphatidylcholine and salts of taurocholic acid, 1,2-diacylphosphatidylcholine and salts of glycocholic acid, and 1,2-diacylphosphatidylcholine and mixtures of salts of taurocholic and glycoholic acid. Preferred embodiments are 1,2-diacylphosphatidylcholine and sodium glycocholate, and 1,2-diacylphosphatidylcholine and sodium taurocholate.

Specific preferred combinations are 1-oleoyl-2-palmitoyl-sn-glycero-3-phosphocholine and sodium taurocholate, and 1-oleoyl-2-palmitoyl-sn-glycero-3-phosphocholine and sodium glycocholate.

The relative amounts of drug, phospholipid and bile salt are important to form primarily nanoparticles rather than liposomes. The weight ratio of the drug. to phospholipid is from 1:0.1 to 1:10, more preferably 1:0.25 to 1:4, and most preferably from 1:0.25 to 1:1. The weight ratio of phospholipid to bile salt is from 1:0.05 to 1:4, more preferably 1:0.1 to 1:2 and most preferably 1:0.125 to 1:0.5.

The amount of drug in the nanoparticle generally ranges from 1 wt % to 80 wt % of the nanoparticle. The drug may constitute at least 5 wt %, 10 wt % 20 wt % or 30 wt % of the nanoparticle. The amount of drug present in the nanoparticle may range from 5 wt % to 70 wt %, preferably from 10 to 60 wt %, and more preferably 20 wt % to 55 wt %.

The drug, phospholipid and bile salt are collectively present in the nanoparticle in an amount ranging from 80 wt % to 100 wt % of the nanoparticle. Preferably, the drug, phospholipid and bile salt constitute at least 85 wt %, more preferably at least 90 wt %, and even more preferably at least 95 wt % of the nanoparticle. In one embodiment, the nanoparticles consist essentially of the non-crystalline drug, phospholipid and bile salt, meaning that the nanopartides contain less than 1 wt % of other materials.

In one embodiment, the amounts of drug, phospholipid and bile salt are:

drug: 5 wt %-70 wt %;

phospholipid: 30 wt %-70 wt %; and

bile salt: 1 wt %-40 wt %.

In another embodiment, the amounts of drug, phospholipid and bile salt are:

drug: 20 wt %-60 wt %;

phospholipid: 40 wt %-60 wt %; and

bile salt: 5 wt % to 30 wt %

The ionizable group(s) on the phospholipid(s) and/or bile salt(s) have a pKa such that they are at least partially ionized at the use conditions. The ionizable groups may be either positively or negatively charged. An indirect measure of the charge is zeta potential. The nanoparticles preferably have ionizable groups sufficient in number to provide a zeta potential in water of less than −10 mV or greater than +10 mV (that is, the absolute value of the zeta potential is greater than 10 mV) under physiologically relevant conditions. Preferably, to reduce aggregation, the absolute value of the zeta potential is at least 25 mV, more preferably at least 40 mV, and even more preferably at least 60 mV. Zeta potential is typically calculated from the electrophoretic mobility measured by light scattering, R. J. Hunter, Zeta Potential in Colloid Science. Principles and Applications, Academic Press, 1981. Zeta potential may be measured in distilled water using any number of commercially-available instruments, such as Brookhaven Instruments Corp. ZetaPals zeta potential analyzer.

### Concentration Enhancement

The nanoparticles improve the concentration of dissolved drug in a use environment relative to a control composition consisting essentially of either (1) the drug alone in bulk crystalline form, or (2) the non-crystalline (or amorphous) form alone if the crystalline form of the drug is unknown. The drug in the control composition is in the form of particles or crystals greater than 1 micron. As used herein, a “use environment” can be the in vivo environment of the GI tract, subdermal, intranasal, buccal, intrathecal, ocular, intraaural, subcutaneous spaces, vaginal tract, arterial and venous blood vessels, pulmonary tract or intramuscular tissue of an animal, such as a mammal and particularly a human, or an in vitro test media such as phosphate buffered saline (PBS) or model fasted duodenal solution (MFDS). By “bulk crystalline form” is meant crystalline drug with a mean particle diameter greater than 1 micron without other solubilizers present.

In order to determine concentration enhancement in vitro, the amount of “free” drug, or solvated drug is measured. By “free” drug is meant drug which is in the form of dissolved drug or present in micelles, but which is not in the nanoparticles.

Several procedures can be used to measure free drug for a nanoparticle suspension. In the filtration procedure, nanoparticles are equilibrated in an aqueous receptor solution, such as water, PBS, or MFDS by stirring. An aliquot of ˜300 μL is withdrawn and placed into a microcentrifuge tube fitted with a 100,000 molecular weight (MW) cutoff filter (regenerated cellulose). The tube is spun at 13000 rpm for 3 minutes, and the filtrate solution is collected. The filtrate solution contains only drug that is dissolved, as the nanoparticles cannot pass through the MW cutoff filter. The drug concentration in the filtrate is analyzed by HPLC.

Alternatively, free drug for a nanoparticle suspension can be measured with nuclear magnetic resonance (NMR). In this method, the nanoparticles are equilibrated in an NMR tube with a buffered deuterium oxide solution. A specified amount of a reference standard is also added to the sample, such that the final concentration of the standard in the tube is known. An NMR spectrum is then acquired, and the integration of the drug peak(s) is compared to that of the reference standard to determine the actual dissolved drug concentration. Depending on the drug, either proton NMR (in which case a suitable reference standard is deuterated trimethylsilyl propionic acid) or fluorine NMR (in which case a suitable reference standard is trifiuoroacetic acid) may be employed. Because NMR is sensitive only to materials in the solution state or in micelles, only the drug that is not sequestered in nanopartides is measured by this method.

Alternatively, the compositions of the present invention, when dosed orally to a human or other animal, provide an area under the drug concentration in the blood plasma or serum versus time curve (AUC; also referred to as relative bioavailability) that is at least 1.25-fold that observed in comparison to the control composition. Preferably, the blood AUC is at least about 2-fold, more preferably at least about 3-fold, even more preferably at least about 4-fold, still more preferably at least about 6-fold, yet more preferably at least about 10-fold, and most preferably at least about 20-fold that of the control composition.

Alternatively, the compositions of the present invention, when dosed orally to a human or other animal, provide a maximum drug concentration in the blood plasma or serum (Cₘₐₓ) that is at least 1.25-fold that observed in comparison to the control composition. Preferably, the Cₘₐₓ is at least about 2-fold, more preferably at least about 3-fold, even more preferably at least about 4-fold, still more preferably at least about 6-fold, yet more preferably at least about 10-fold, and most preferably at least about 20-fold that of the control composition. Thus, compositions that meet the in vitro or in vivo performance criteria, or both, are considered to be within the scope of the invention.

Relative bioavailability or Cₘₐₓ of drugs in the compositions of the invention can be tested in vivo in animals or humans using conventional methods for making such a determination, such as a crossover study. In an exemplary in vivo crossover study, a test composition comprising the nanoparticles is dosed to half a group of test subjects and, after an appropriate washout period (e.g., one week) the same subjects are dosed with a control composition that consists of an equivalent quantity of crystalline drug as was dosed with the test composition, but with no dispersion polymer present. The other half of the group is dosed with the control composition first, followed by the test composition. Relative bioavailability is measured as the concentration of drug in the blood (serum or plasma) versus time AUC determined for the test group divided by the AUC in the blood provided by the control composition. Preferably, this test/control ratio is determined for each subject, and then the ratios are averaged over all subjects in the study. In vivo determinations of AUC and Cₘₐₓ can be made by plotting the serum or plasma concentration of drug along the ordinate (y-axis) against time along the abscissa (x-axis). To facilitate dosing, a dosing vehicle may be used to administer the dose. The dosing vehicle is preferably water or a buffer with no surfactants, but may also contain materials for suspending the test or control composition, provided these materials do not change the aqueous solubility of the drug in vivo. The determination of AUCs is a well-known procedure and is described, for example, in Welling, “Pharmacokinetics Processes and Mathematics,” ACS Monograph 185 (1986).

### Process for Forming Nanoparticles

The nanoparticles may be formed by any process that results in formation of nanoparticles of non-crystalline drug and with phospholipid(s) and bile salt(s) as surface stabilizers.

One process to form nanoparticles is an emulsification process. In this process, the drug is dissolved in an organic solvent that is immiscible with a non-solvent for the drug. The surface stabilizer(s) may be dissolved in either the organic solvent, the non-solvent, or both. Typically, the phospholipid(s) are dissolved in the organic solvent and the bile salts) are dissolved in the non-solvent. The organic solvent solution is added to the non-solvent solution and homogenized to form an emulsion of fine droplets of the water immiscible solvent phase distributed throughout the non-solvent phase. The solvent is then evaporated to form nanoparticles in the non-solvent phase. Exemplary solvents include methylene chloride, trichloroethylene, trichlorotrifluoroethylene, tetrachloroethane, trichloroethane, dichloroethane, dibromoethane, ethyl acetate, phenol, chloroform, toluene, xylene, ethyl-benzene, benzyl alcohol, creosol, methyl-ethyl ketone, methyl-isobutyl ketone, hexane, heptane, ether, and mixtures thereof. Preferred organic solvents for use in such a process include methylene chloride, ethyl acetate, cyclohexane, and benzyl alcohol. Exemplary non-solvents for the drug include water.

The emulsion is generally formed by a two-step homogenization procedure. The solution of drug, surface stabilizers and solvent and the non-solvent are first mixed with a rotor/stator or similar mixer to create a “pre-emulsion”. This mixture is then further processed with a high pressure homogenizer that subjects the droplets to very high shear, creating a uniform emulsion of very small droplets. A portion of the solvent is then removed forming a suspension of the nanoparticles in the non-solvent. Exemplary processes for removing the solvent include evaporation, extraction, diafiltration, pervaporation, vapor permeation, distillation, and filtration.

In order to form stable nanopartides of solid non-crystalline drug, it is necessary to have a sufficient amount of bile salt present relative to the droplets of organic solvent (non-solvent immiscible phase). The ratio of bile salt to organic solvent (non-solvent immiscible phase) in the emulsion is 0.1 mg/ml to 100 mg/ml, more preferably 1 to 50 mg/ml. The ratio of the organic solvent (non-solvent immiscible phase) to the non-solvent is 1 ml organic solvent/100 ml aqueous to 70 ml organic solvent/100 ml non-solvent, and more preferably from 10 ml organic solvent/100 ml non-solvent to 50 ml organic solvent/100 ml non-solvent.

A variety of processes may be used to form solid compositions comprising the nanoparticles. Essentially any process that removes the liquid from the suspension may be used to form a solid composition, provided the process does not affect the properties of the nanoparticles. Exemplary processes include spray drying, spray coating, spray layering, lyophylization, evaporation, vacuum evaporation, and filtration. A preferred process is spray drying. One or more processes may be combined to remove the liquid from the nanoparticle suspension and yield a solid composition. For example, a portion of the solvent and non-solvent may be removed by filtration to concentrate the nanopartides, followed by spray-drying to remove most of the remaining solvent and non-solvent, followed by a further drying step such as tray-drying. Removal of the liquid results in solid nanoparticles of solid non-crystalline drug with surface stabilizers consisting of phospholipid(s) and bile salt(s).

Excipients may be added to the aqueous suspension containing the nanoparticles prior to removal of the liquid to form the solid composition. One such excipient is a matrix material. Exemplary matrix materials include acacia, trehalose, lactose, mannitol and casein, and pharmaceutically acceptable forms thereof. Casein, caseinate, and pharmaceutically acceptable forms thereof are preferred matrix materials. In this embodiment, the nanoparticles are entrapped in the matrix material.

### Dosage Forms

The nanoparticles may be administered using any known dosage form. The nanoparticles may be formulated for oral, subdermal, intranasal, buccal, intrathecal, ocular, intraaural, subcutaneous spaces, vaginal tract, arterial and venous blood vessels, pulmonary tract or intramuscular tissue of an animal, such as a mammal and particularly a human. Oral dosage forms include: powders or granules; tablets; chewable tablets; capsules; unit dose packets, sometimes referred to in the art as “sachets” or “oral powders for constitution” (OPC); syrups; and suspensions. Parenteral dosage forms include reconstitutable powders or suspensions. Topical dosage forms include creams, pastes, suspensions, powders, foams and gels. Ocular dosage forms include suspensions, inserts, and gels.

Without further elaboration, it is believed that one of ordinary skill in the art can, using the foregoing description, utilize the present invention to its fullest extent. Therefore, the following specific embodiments are to be construed as merely illustrative and not restrictive of the scope of the invention. Those of ordinary skill in the art will understand that variations of the conditions and processes of the following examples can be used.

### EXAMPLES

### Examples 1-3

Surface stabilized nanoparticles containing the CETP inhibitor [2R,4S] 4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester (“Drug 1”, also known as torcetrapib) were prepared. Drug 1 has a T_{g} of 30° C., a Tₘ of 95° C., and a Log P of about 7.55. For the nanoparticles of Example 1, 150 mg torcetrapib and 150 mg of the phospholipid 1,2-diacylphosphatidylcholine (from egg yolk, Type XVI-E, approx. 99%, available from Sigma, St. Louis, Mo.) (“PPC”) were dissolved in 3 mLs methylene chloride to form an organic solution. Next, 18 mg of the bile salt sodium glycocholate (also available from Sigma) (“NaGC”) was dissolved in 34.5 mL deionized water to form an aqueous solution. The organic solution was then poured into the aqueous solution and emulsified for 3 minutes using a Kinematica Polytron 3100 rotor/stator at 10,000. rpm. The solution was further emulsified to reduce particle size using a Microfluidizer (Microfluidics model M-110L F12Y with Z chamber, ice bath and cooling coil), 100 passes at 12,500 psi. The emulsion was then stirred for 3 hours at room temperature in a fume hood to evaporate the methylene chloride. The nanoparticles of Examples 2 and 3 were made using the procedures described above, with the compositions shown in Table 1. For Example 3, the bile salt sodium taurocholate (“NaTC”) was used instead of sodium glycocholate.

TABLE 1

| Example | | Formulation |
|---|---|---|
| No. | Drug | (wt:wt:wt) |
| 1 | Torcetrapib | 8:8:1 drug:PPC:NaGC |
| | (Drug 1) | |
| 2 | Torcetrapib | 4:4:1 drug:PPC:NaGC |
| | (Drug 1) | |
| 3 | Torcetrapib | 2:4:1 drug:PPC:NaTC |
| | (Drug 1) | |

### Dynamic Light Scattering Analysis

For dynamic light scattering (DLS) analysis, the suspensions above (after evaporation) were filtered using a 0.2 μm Steriflip® filter (Millipore Corp., Billerica, Mass.). A cuvette was filled with 0.5 mg/mL sodium glycocholate in deionized water, and 50 μL of the filtered emulsion solution was added. Dynamic light-scattering was measured using a Brookhaven Instruments BI-200SM particle size analyzer with a BI-9000AT correlator (Brookhaven Instruments, Holtsville, N.Y.). The size is reported as the cumulant value, shown in Table 2.

The size was measured again after 24 hours to evaluate particle agglomeration in solution. The results showed that particles did not significantly agglomerate after 24 hours, indicating that the suspension was stable. The particle sizes are shown in Table 2.

TABLE 2

| | Suspension following | | | |
|---|---|---|---|---|
| | solvent evaporation | | Suspension after 24 hrs | |
| Example No | Size (nm) | Polydispersity | Size (nm) | Polydispersity |
| 1 | 66 | 0.12 | 79 | 0.22 |
| 2 | 39 | 0.25 | 41 | 0.26 |
| 3 | 65 | 0.47 | — | — |

### Zeta Potential Analysis

The zeta potential of the aqueous suspension of Example 2 was analyzed without further processing. The Brookhaven Instruments BI-200SM particle size analyzer was equipped with a ZetaPALS (Brookhaven Instruments, Holtsville, N.Y.) analyzer to measure zeta potential. The ZetaPALS analyzer utilizes phase analysis light scattering to determine the electrophoretic mobility of charged, colloidal suspensions. For the aqueous suspension of Example 2, the zeta potential was found to be −25 mV.

### Control 1

### Nanoparticles without Bile Salt

Nanoparticles containing torcetrapib were prepared using procedures described above for Example 1, except that they did not contain sodium glycocholate. For the nanoparticles of Control 1, 100 mg torcetrapib and 100 mg PPC were dissolved in 2 mLs methylene chloride, and this solution was poured into 23 mL deionized water. The emulsion was formed as described above, and the solvent was evaporated in a fume hood.

### Dynamic Light Scattering Analysis

The nanoparticles of Control 1 were characterized using DLS analysis as described above except that the suspension was not filtered prior to analysis. The cumulant particle size was found to be 295 nm, and visible precipitate was observed. These results show that the nanopartides of Control 1 without bile salt agglomerate.

### Isolation of Solid Nanoparticles

To isolate the nanopartides of Example 2 in dried powder form, trehalose was added to the suspension above (1% trehalose wt/suspension mL). The suspension was filtered using a 0.22 μm Steriflip® filter, then lyophilized overnight to obtain a powder containing Drug 1:PPC:NaGC:trehalose in a ratio of 4:4:1:9.

### Differential Scanning Calorimetry

The dried nanoparticles of Example 2 were analyzed using modulated differential scanning calorimetry (MDSC). The sample pans were crimped and sealed at ambient temperature and humidity, then loaded into a Thermal Analysis Q1000 DSC equipped with an autosampler. The samples were heated by modulating the temperature at ±1.5° C./min, and ramping at 2.5° C./min to 175° C. The glass transition temperature of the nanoparticles of Example 2 was determined to be 26° C. from the DSC scans.

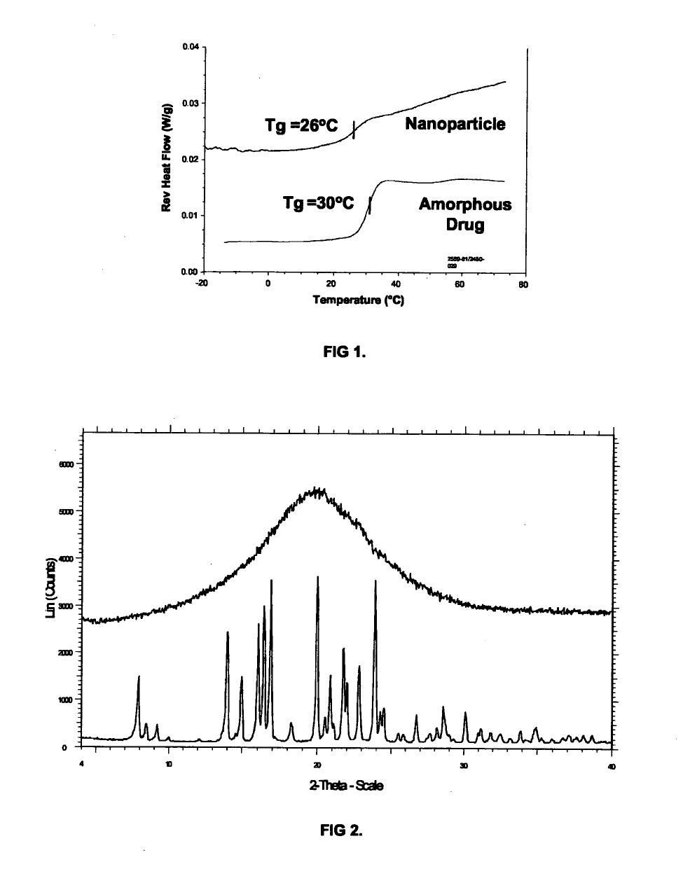

The results are shown below in FIG. 1. Pure non-crystalline (or amorphous) Drug 1 is also shown for comparison. The DSC results indicate that at least some of Drug 1 in the nanoparticles of Example 2 is in the non-crystalline form.

### PXRD Evaluation

The nanoparticles of Example 2 were examined using powder x-ray diffraction (PXRD) with a Bruker AXS D8 Advance diffractometer to determine the crystalline character of the drug in the nanoparticles. Samples (approximately 100 mg) were packed in Lucite sample cups fitted with Si(511) plates as the bottom of the cup to give no background signal. Samples were spun in the φ plane at a rate of 30 rpm to minimize crystal orientation effects. The x-ray source (KCu_{α}, λ=1.54 Å) was operated at a voltage of 45 kV and a current of 40 mA. Data for each sample were collected over a period of 36 minutes in continuous detector scan mode at a scan speed of 1.8 seconds/step and a step size of 0.04°/step. Diffractograms were collected over the 2θ range of 4° to 40°. FIG. 2 is a diffraction pattern of the nanoparticles of Example 2 showing only an amorphous halo, instead of a pattern showing sharp peaks characteristic of crystalline drug. These data indicate that the drug in the nanoparticles of Example 2 is in the non-crystalline (or amorphous) form.

### Measurement of Free Drug

The amount of free drug provided by the nanoparticles of Example 2 was measured. “Free drug” refers to drug molecules which are dissolved in the aqueous solution and are generally either monomeric or dusters of no more than 100 molecules. The amount of free drug provided by the nanoparticles of Example 2 was measured using nuclear magnetic resonance (NMR). For this test, a sample of the dried nanoparticles of Example 2 was added to a centrifuge tube containing deuterated PBS with 2.0 wt % 4/1 sodium taurocholic acid/1-palmitoyl-2-oleyl-sn-glycero-3-phosphocholine (“NaTC/POPC”). The solution was mixed, and an ¹⁹F internal standard solution of trifluoroacetic acid (TFA) was added. The Drug 1 concentration would have been 1000 μg/mL if all of the sample dissolved. This solution was vortexed 1 minute, and then carefully transferred to an 8 mm glass NMR tube.

Fluorine spectra of the sample was recorded at 282.327 MHz on a Varian Gemini 2000, 300 MHz NMR equipped with a Nalorac 8 mm indirect detection probe. The sample temperature was maintained at 37° C. in the probe. Drug resonances were integrated relative to the internal standard peak and the drug concentration determined.

The concentration of free drug measured after 120 minutes is shown in Table 3. Crystalline Drug 1 having a particle size of from 83 to 588 μm is shown for comparison. The concentration of free drug provided by the nanoparticle suspension of Example 2 is 3.3-fold the concentration of free drug provided by Crystalline Drug 1. The higher free drug concentration is expected to result in greater bioavailability of Drug 1 in vivo.

TABLE 3

| | Free Drug |
|---|---|
| Nanoparticle Suspension | (μg/mL) |
| Example 2 | 50.1 |
| 4:4:1 Drug 1:PPC:NaGC | |
| Crystalline Drug 1 | 15 |

### In Vivo Evaluation in Dogs

The nanoparticles of Example 2 were evaluated in vivo in dogs. Samples were dosed orally as a suspension to 6 male beagle dogs.

Animals were fasted overnight (at least 10 hours predose) through 8 hours postdose. Approximately 50 mL of suspension (1.2 mgA/mL) was administered to each dog via oral gavage, followed by approximately 5 mL water. Whole-blood samples (7-mL sodium heparin Vacutainer tubes) were taken from the jugular vein before dosing and at 0.5, 1, 2, 3, 4, 6, 8, 12, 24, and 28 hours after dosing. Serum was harvested into tubes containing K₂EDTA anticoagulant. Blood was maintained on wet ice prior to centrifugation to obtain plasma. Centrifugation began within 1 hour of collection, and samples were centrifuged at 2500 rpm for 15 minutes. Plasma was maintained on dry ice prior to storage at approximately −70° C. Plasma was analyzed using liquid chromatography with tandem mass spectrometry (LC/MS/MS). The results are shown in Table 4. Crystalline Drug 1 was tested for comparison.

TABLE 4

| | crystalline | |
|---|---|---|
| Parameter | Drug 1 | Example 2 |
| Cₘₐₓ (ng/mL) | <LOQ* | 870 |
| Tₘₐₓ (hours) | <LOQ | 0.67 |
| AUC_{0-∞} (ng/mL-hr) | <LOQ | 2700 |

*<LOQ = less than limit of quantitation

The drug concentration in vivo provided by crystalline drug could not be measured. The nanoparticles of Example 2 provide significant solubilization of Drug 1 in vivo.

### Example 4

Surface stabilized nanoparticles containing the CETP inhibitor [2R,4S] 4-[acetyl-(3,5-bis-trifluoromethyl-benzyl)-amino]-2-ethyl-6-trifluoromethyl-3,4-dihydro-2H-quinoline-1-carboxylic acid isopropyl ester (“Drug 2”) were prepared as described above for Example 1. Drug 2 has a T_{g} of 45° C., a Tₘ of 111° C., and a Log P of about 7.55. The nanoparticle formulation of Example 4 contained Drug 2, PPC, and NaGC in a mass ratio of 8:8:1.

### Isolation of Solid Nanoparticles

Spray-drying was used to isolate dried nanoparticles of the invention. Following evaporation of methylene chloride from the emulsion, 3.125 g trehalose was added to 62.5 g Example 4 nanoparticle solution. The solution was pumped into a “mini” spray-drying apparatus via a Cole Parmer 74900 series rate-controlling syringe pump at a rate of 6 ml/hr. The drug/polymer solution was atomized through a Spraying Systems Co. two-fluid nozzle, Model No. SU1A using a heated stream of nitrogen at a flow rate of 1 SCFM. The spray solution was sprayed into an 11-cm diameter stainless steel chamber. The heated gas entered the chamber at an inlet temperature of 120° C. and exited at an outlet temperature of 22° C.

### Dynamic Light Scattering Analysis

The spray-dried nanoparticles of Example 4 were resuspended by adding 54.8 mg to 4 mLs 5 wt % dextrose in water solution to obtain a drug concentration of about 1 mg/mL. The solution was filtered through a 1 μm glass membrane filter (Anatop filter, Whatman), and analyzed using DLS immediately following resuspension and after 24 hours. Immediately following resuspension the cumulant nanoparticle size was found to be 124 nm, with a polydispersity of 0.24, and 24 hours after resuspension the cumulant nanoparticle size was found to be 153 nm, with a polydispersity of 0.64.

### Examples 5 and 6

Surface stabilized nanoparticles containing Drug 2 were prepared as described above for Example 1, with the exceptions noted in Table 5. The nanoparticle formulation of Example 5 contained Drug 2, PPC, and NaTC in a mass ratio of 1:2:1. The nanoparticle formulation of Example 6 contained Drug 2, PPC, and NaTC in a mass ratio of 8:8:1.

TABLE 5

| | | | Methylene | | | | | Rotary |
|---|---|---|---|---|---|---|---|---|
| | Drug 2 | PPC | Chloride | NaTC | Water | Rotosator | Homogenizer | Evaporation |
| Example | (mg) | (mg) | (mL) | (mg) | (mL) | (min) | (min) | (min) |
| 5 | 125 | 250 | 5 | 125 | 50 | 5 | 5 | 30 |
| 6 | 100 | 100 | 2 | 12.5 | 23 | 4 | not recorded | 30 |

The nanoparticles of Example 6 were characterized using DLS analysis, and had a cumulant size of 62 nm with a polydispersity of 0.13, following formation, and a size of 81 nm with a polydispersity of 0.36 24 hours after formation.

### Concentration Enhancement

### In Vitro Dissolution Tests

An in vitro dissolution test was used to determine the dissolution performance of the nanoparticles of Examples 5 and 6. For this test, a sufficient amount of material was added to a scintillation vial so that the concentration of Drug 2 would have been 40 μgA/mL, if all of the drug had dissolved. The test was run in duplicate. The vials were placed in a 37° C. temperature-controlled chamber, and 100 μL suspension was added to 4.9 mL PBS at pH 6.5 and 290 mOsm/kg, containing 2.0 wt % NaTC/POPC. The samples were quickly mixed using a vortex mixer for about 30 seconds. The samples were centrifuged using Microcon YM-100 centrifuge filters at 12,000 G at 37° C. for 5 minutes. The resulting supernatant solution was then sampled and diluted 1:5 (by volume) with methanol and analyzed by HPLC. The contents of each tube were mixed on the vortex mixer and allowed to stand undisturbed at 37° C. until the next sample was taken. Samples were collected at 5, 10, 15, 20, 30, 60, and 1200 minutes. Table 6 shows the maximum drug concentration within sixty minutes (MDC₆₀) achieved during dissolution testing, and the drug concentration after 1200 minutes (C₁₂₀₀).

TABLE 6

| | MDC₆₀ | C₁₂₀₀ |
|---|---|---|
| sample | (μg/mL) | (μg/mL) |
| Example 5 | 24 | 21 |
| 1:2:1 Drug 2:PPC:NaTC | | |
| Example 6 | 23 | 21 |
| 8:8:1 Drug 2:PPC:NaTC | | |

The results show that the nanoparticles of Examples 5 and 6 provide concentration-enhancement of Drug 2 relative to the solubility of crystalline Drug 2 in 2 wt % NaTC/POPC (approximately 8 μg/mL). In addition, the increased Drug 2 concentration was maintained for at least 1200 minutes, without agglomeration of the nanoparticles in suspension.

### Measurement of Free Drug

The amount of free drug provided by the nanoparticle suspensions of Examples 5 and 6 was measured as described above. For NMR analysis of the suspensions, 500 μL of the suspension was added to 500 μL of deuterated PBS containing the TFA internal standard and 200 mg NaTC/POPC. The concentration of free drug measured is shown in Table 7 Crystalline Drug 2 is shown for comparison. The concentration of free drug provided by the nanoparticle suspension of Example 5 is 4.3-fold the concentration of free drug provided by Crystalline Drug 2. The concentration of free drug provided by the nanoparticle suspension of Example 6 is 4.4-fold the concentration of free drug provided by Crystalline Drug 2. The higher free drug concentrations are expected to result in greater bioavailability of Drug 2 in vivo.

TABLE 7

| | Free Drug |
|---|---|
| Nanoparticle Suspension | (μg/mL) |
| Example 5 | 34 |
| 1:2:1 Drug 2:PPC:NaTC | |
| Example 6 | 35 |
| 8:8:1 Drug 2:PPC:NaTC | |
| Crystalline Drug 2 | 8 |

### In Vivo Evaluation in Rats

The nanoparticle suspension of Example 5 was tested in vivo in Sprague-Dawley rats (n=5).

The aqueous nanoparticle suspension (2 mg/mL potency; 10 mg/kg dose) was administered orally to fed or fasted rats via a ball-tipped gavage needle. Approximately 0.3 mL blood samples were collected predose, and at 0.25, 0.5, 1, 2, 3, 4, 5, 6, 8, 12, and 24 hours postdose. Blood was collected from a jugular vein via syringe and needle and transferred into tubes containing K₂EDTA anticoagulant. Blood was maintained on wet ice prior to centrifugation to obtain plasma. Centrifugation began within 1 hour of collection, and samples were centrifugation at 2500 rpm for 15 minutes. Plasma was maintained on dry ice prior to storage at approximately −70° C. Plasma was analyzed using liquid chromatography with tandem mass spectrometry (LC/MS/MS). The results are shown in Table 8.

TABLE 8

| | Example 5 | Example 5 |
|---|---|---|
| Parameter | fed | fasted |
| Cₘₐₓ (ng/mL) | 818 | 479 |
| Tₘₐₓ (hours) | 5.8 | 1.8 |
| AUC_{0-∞} (ng/mL-hr) | 7944 | 2905 |

### Examples 7, 8, and 9

Surface stabilized nanoparticles containing Drug 2 were prepared as described above for Example 1, with the exceptions noted in Table 9. The nanoparticle formulation of Example 7 contained Drug 2, PPC, and NaTC in a ratio of 1:2:1. The nanoparticle formulation of Example 8 contained Drug 2, PPC, and NaTC in a ratio of 8:8:1. The nanoparticle formulation of Example 9 contained Drug 2, PPC, and NaTC in a ratio of 1:2:1.

TABLE 9

| | | | Methylene | | | | | Rotary |
|---|---|---|---|---|---|---|---|---|
| | Drug 2 | PPC | Chloride | NaTC | Water | Rotosator | Homogenizer | Evaporation |
| Example | (g) | (g) | (mL) | (mg) | (mL) | (min) | (min) | (min) |
| 7 | 0.5 | 1 | 20 | 500 | 200 | 10 | 10 | 30 |
| 8 | 0.6 | 0.6 | 12 | 75 | 120 | 4 | not recorded | 30 |
| 9 | 0.5 | 1 | 5 | 500 | 200 | 10 | 20 | 25 |

The nanoparticles of Examples 7-9 were characterized using DLS analysis, and the cumulant sizes are reported in Table 10.

TABLE 10

| | Suspension | | | |
|---|---|---|---|---|
| | following solvent | | Suspension | |
| | evaporation | | after 24 hrs | |
| Example No | Size (nm) | Polydispersity | Size (nm) | Polydispersity |
| 7 | 75 | 0.52 | 126 | 0.43 |
| 8 | 46 | 0.38 | 285 | 0.61 |
| 9 | 148 | 0.25 | 147 | 0.23 |

### In Vivo Evaluation in Dogs

The nanoparticles of Examples 7, 8, and 9 were evaluated in vivo in dogs. Samples were dosed orally as a suspension to 6 male beagle dogs. Animals were fasted overnight (at least 10 hours predose) through at least 12 hours postdose. For Examples 7 and 8, approximately 50 mL of 2 mg/mL suspension (10 mgA/kg) was administered to each dog. For Example 9, approximately 50 mL of 0.04, 0.2, or 0.8 mg/mL suspension (0.2, 1, or 4 mgA/kg) was administered to each dog. Each dose was administered via oral gavage, followed by approximately 5 mL water. Whole-blood samples were taken from the jugular vein before dosing and at 0.25, 0.5, 1, 2, 3, 4, 5, 6, 8, 12, and 24 hours after dosing. Serum was harvested into tubes containing K₂EDTA anticoagulant. Blood was maintained on wet ice prior to centrifugation to obtain plasma. Centrifugation began within 1 hour of collection, and samples were centrifugation at 2500 rpm for 15 minutes. Plasma was maintained on dry ice prior to storage at approximately −70° C. Plasma was analyzed using liquid chromatography with tandem mass spectrometry (LC/MS/MS). The results are shown in Table 11.

TABLE 11

| | Example 7 | Example 8 | Example 9 | | |
|---|---|---|---|---|---|
| | 10 mgA/ | 10 mgA/ | 0.2 mgA/ | | |
| Parameter | kg | kg | kg | 1 mgA/kg | 4 mgA/kg |
| Cₘₐₓ | 579 | 422 | 18 | 90 | 371 |
| (ng/mL) | | | | | |
| Tₘₐₓ (hours) | 1.17 | 1.08 | 1.17 | 0.917 | 1.00 |
| AUC_{0-∞} | 2138 | 2081 | 41 | 219 | 850 |
| (ng/mL-hr) | | | | | |

### Example 10

### Emulsion Formation

Surface stabilized nanoparticles containing the CETP inhibitor [2R,4S] 4-[(3,5-bis-trifluoromethyl-benzyl)-methoxycarbonyl-amino]-2-ethyl-6-trifluoromethyl-3,4-dihydro-2H-quinoline-1-carboxylic acid isopropyl ester (“Drug 3”) were prepared as described above for Example 1. Drug 3 has a T_{g} of 29° C., a Tₘ of 91° C. and a calculated Log P of 7.86. For the nanoparticles of Example 10, 100 mg Drug 3 and 100 mg PPC were dissolved in 2 mL methylene chloride, and 12.5 mg NaGly was dissolved in 23 mL deionized water. The nanoparticle formulation of Example 10 contained Drug 3, PPC, and NaGC in a ratio of 8:8:1.

### Isolation of Solid Nanoparticles

To obtain the dried nanoparticles of Example 10, 0.5 g trehalose was added to 9.3 g of the emulsion above. The solution was filtered using a 0.22 μm Steriflip® filter, then lyophilized overnight to obtain a dry powder.

### Dynamic Light Scattering Analysis

The nanoparticles of Example 10 were characterized using DLS analysis as described above. The mean particle size was found to be 112 nm immediately after formation of the nanoparticle suspension, 86 nm with a polydispersity of 0.33 after 24 hours in suspension, and 105 nm with a polydispersity of 0.13 following resuspension of the dried powder. These results show that small, stable nanoparticles of the invention can be formed using Drug 3.

### Control 2

Nanoparticles were made containing linezolid ((S)-N-[[3-[3-Fluoro-4-(4-morpholinyl)phenyl]-2-oxo-5-oxazolidinyl]methyl]-acetamide). Linezolid has a melting point of 75° C., and a logP of 0.5. Linezolid is outside of the scope of this invention. For the nanoparticles of Control 2, 502 mg linezolid and 504 mg PPC were dissolved in 16 mLs methylene chloride, and this solution was poured into 40 mL deionized water containing 125 mg NaGC. The emulsion was formed as described above, and the solvent was evaporated in a fume hood.

### Optical Microscopy Analysis

The nanoparticles of Control 2 were characterized using optical microscopy (Nikon Eclipse E-600 microscope with camera and Clemex ST-2000 image software). Visual observations showed particle aggregation and precipitation from solution. Drug crystals were observed in microscope images.

### Control 3

Nanoparticles were made containing chloramphenicol (acetamide, 2,2-dichloro-N-[2-hydroxy-1-(hydroxymethyl)-2-(4-nitrophenyl)ethyl]-[R-(R*,R*)]-). Chloramphenicol has a melting point of 150° C., and a logP of −0.23. Chloramphenicol is outside the scope of this invention. For the nanoparticles of Control 3, 300 mg chloramphenicol and 300 mg PPC were dissolved in 9 mLs methylene chloride, and this solution was poured into 20 mL deionized water containing 75 mg NaGC. The emulsion was formed as described above, and the solvent was evaporated in a fume hood.

### Optical Microscopy Analysis

The nanoparticles of Control 3 were characterized using optical microscopy. Visual observations showed particle aggregation and precipitation from solution. Drug crystals were observed in microscope images.

### Control 4

Nanoparticles were made using the procedures described in the following paper “A Method for the Preparation of Submicron Particles of Sparingly Water-Soluble Drugs by Precipitation in Oil-in-Water Emulsions. II: Influence of the Emulsifier, the Solvent, and the Drug Substance”; Brita Sjöström, et al., Institute for Surface Chemistry, Oct. 13, 1992. PXRD was used to characterize the model drug in the nanoparticles. Cholesterol acetate has a T_{g} of −12° C., a Tₘ of 112° C. and a calculated LogP of 7.6, which is outside the scope of this invention.

### Emulsion Formation

Nanoparticles containing cholesteryl acetate were prepared as follows. First, 752.8 mg cholesteryl acetate and 120.7 mg L, α-phospatidylcholine (“PPC”) were dissolved in 3.0285 g cyclohexane to form an organic solution. Next, 30.8 mg sodium glycocholate (“NaGC”) was dissolved in 27.0184 g deionized water to form an aqueous solution. The organic solution was then poured into the aqueous solution and emulsified for 4 min using a Kinematica Polytron 3100 rotor/stator at 10,000 rpm. The solution was further emulsified for 4 min using. a Microfluidizer (Microfluidics model M-110L F12Y with Z chamber, ice bath and cooling coil). Solvent was removed using a rotary evaporator for 10 minutes at 200 rpm and 30° C.

### Cryo-Transmission Electron Microscope Analysis

The nanoparticles were evaluated using cryo-transmission electron microscopy (TEM). A Tecnai G² series TEM (FEI Company; Hillsboro, Oreg.) was used to observe the fine structure morphology of nanoparticles in the solution above, following the rotary evaporation step. Electron diffraction spot patterns showed that a large portion of the drug was present in crystalline form in the nanoparticles.

### Dynamic Light Scattering Analysis

For dynamic light scattering (DLS) analysis, a cuvette was filled with deionized water, and 3 drops of the suspension were added. Dynamic light-scattering was measured using a Brookhaven Instruments BI-200SM particle size analyzer with a BI-9000AT correlator. The sums of exponentials from the autocorrelation functions are analyzed to extract size distributions from the samples. The cumulant diameter (average of two samples) was found to be 72 nm, with a polydispersity of 0.23.

### Isolation of Solid Nanoparticles

To isolate the nanoparticles in dried powder form, the suspension was lyophilized overnight.

### PXRD Evaluation

The dried nanoparticles were examined using powder x-ray diffraction (PXRD) with a Bruker AXS D8 Advance diffractometer to determine the crystalline/amorphous character of the cholesteryl acetate in the nanoparticles. Samples (approximately 100 mg) were packed in Lucite sample cups fitted with Si(511) plates as the bottom of the cup to give no background signal. Samples were spun in the φ plane at a rate of 30 rpm to minimize crystal orientation effects. The x-ray source (KCu_{α}, λ=1.54 Å) was operated at a voltage of 45 kV and a current of 40 mA. Data for each sample were collected over a period of 27 minutes in continuous detector scan mode at a scan speed of 1.8 seconds/step and a step size of 0.04°/step. Diffractograms were collected over the 2θ range of 4° to 40°. PPC, sodium glycocholate, and cholesterol acetate were also examined for comparison. The nanoparticles exhibited a diffraction pattern showing sharp peaks characteristic of crystalline cholesteryl acetate.

The terms and expressions which have been employed in the foregoing specification are used therein as terms of description and not of limitation, and there is no intention in the use of such terms and expressions of excluding equivalents of the features shown and described or portions thereof, it being recognized that the scope of the invention is defined and limited only by the claims which follow.

1. A pharmaceutical composition comprising nanoparticles, said nanoparticles comprising:
(a) a poorly water soluble drug having a solubility in an aqueous solution of pH 6.5 to 7.5 of less than 1 mg/ml, at least 90 wt % of said drug in said composition being non-crystalline;
(b) phospholipid and bile salt present in a weight ratio of from 1:0.05 to 1:4 (wt phospholipid:wt bile salt);
(c) said nanoparticles having an average size of less than 500 nm;
(d) said drug, said phospholipid, and said bile salt collectively constituting at least 80 wt % of said nanopartides;
(e) said nanoparticles comprising a core comprising said drug surrounded by a layer comprising said phospholipid and said bile salt; and
wherein said drug has a LogP of greater than 4 and at least one of (i) a melting temperature (Tₘ) of less than 110° C. and (ii) a glass transition temperature (T_{g}) of greater than 40° C.

2. The composition of claim 1 wherein said T_{g} of said drug is greater than 50° C.

3. The composition of claim 1 wherein said Tₘ of said drug is less than 100° C.

4. The composition of claim 1 wherein said drug has a ratio of Tₘ (in K) to T_{g} (in K) of less than 1.35 (K/K).

5. The composition of claim 1 wherein said drug has a Log P of greater than 5.

6. The composition of claim 1 wherein said core is at least 75 wt % said drug.

7. The composition of claim 1 wherein said weight ratio of phospholipid:bile salt is from 1:0.1 to 1:2 (wt phospholipid:wt bile salt).

8. The composition of claim 1 wherein said drug:phospholipid weight ratio is from 1:0.1 to 1:10.

9. The composition of claim 1 wherein said nanoparticles have an average size of less than 300 nm.

10. The composition of claim 1 wherein said nanoparticles have a zeta potential with an absolute value of greater than 10 mV.

11. The composition of claim 1 wherein said drug constitutes from 5 wt % to 70 wt % of said nanoparticles.

12. The composition of claim 1 wherein said nanoparticles consist essentially of said drug, said phospholipid and said bile salt.

13. The composition of claim 1 comprising 20 wt % to 60 wt % said drug, 40 wt % to 60 wt % said phospholipid and 5 wt % to 30 wt % said bile salt.

14. The composition of claim 1 wherein said bile salt is selected from the group consisting of salts of taurocholic acid, salts of glycocholic acid, and mixtures thereof.

15. The composition of claim 1 wherein said drug is a cholesterol ester transfer protein inhibitor.

16. The composition of claim 15 wherein said drug has a log P of greater than 4 and a Tₘ/T_{g} of less than 1.35.

17. A process for forming nanoparticles, comprising:
(a) dissolving a poorly water soluble drug in an organic solvent to form a solution, wherein said drug has a solubility in aqueous solution of pH 6.5 to 7.5 of less than 1 mg/ml, said drug has a Log P greater than 4, and at least one of a melting temperature (Tₘ) of less than 110° C. and a glass transition temperature (T_{g}) greater than 40° C.;
(b) forming an emulsion comprising said solution and a non-solvent, said drug being poorly soluble in said non-solvent and said solvent being immiscible in said non-solvent, and phospholipid and bile salt present in a weight ratio of from 1:0.05 to 1:4 (wt phospholipid:wt bile salt);
(c) removing at least a portion of said organic solvent to form a suspension of solid nanoparticles having an average size of less than 500 nm, wherein at least 90 wt % of said drug in said nanoparticles is non-crystalline, said drug, said phospholipid, and said bile salt constitute at least 80 wt % of said nanoparticles, and said nanoparticles comprise a core comprising said drug surrounded by a layer comprising said phospholipid and said bile salt.

18. The process of claim 17 wherein said organic solvent is selected from the group consisting of methylene chloride, ethyl acetate, cyclohexane and benzyl alcohol.

19. The process of claim 17 wherein said non-solvent is water.

20. The process of claim 17 wherein said solvent solution and said non-solvent are first combined to form a pre-emulsion and then homogenized to form said emulsion.
